(19) United States
(12) Patent Application Publication
(10) Pub. No.: US 2018/0268925 A1
(43) Pub. Date: Sep. 20, 2018

(54) METHOD FOR INTEGRATING DIAGNOSTIC DATA

(71) Applicant: CHANG GUNG MEMORIAL HOSPITAL, LINKOU, Taoyuan City (TW)

(72) Inventors: Pao-Hsien CHU, Taoyuan City (TW); Ben-Chang SHIA, Taipei City (TW)

(21) Appl. No.: 15/868,908

(22) Filed: Jan. 11, 2018

(30) Foreign Application Priority Data
Mar. 15, 2017 (TW) 106108490

(51) Int. Cl. G16H 10/60; G06F 17/30

(52) U.S. Cl. CPC G16H 10/60; G06F 17/30371

(57) ABSTRACT

A method for integrating diagnostic data, includes steps of: reading a plurality of medical records each including an identity information and a medical history that corresponds to the identify information from a first database and a second database; determining whether any one of the medical records satisfies a user defined condition; and, when affirmative, generating an integrated medical information indicating each of the medical records that satisfies the user defined condition.

### CROSS-REFERENCE TO RELATED APPLICATION

This application claims priority to Taiwanese Patent Application No. 106108490 filed on Mar. 15, 2017.

### FIELD

The disclosure relates to a method for integrating data, more particularly to a method for integrating diagnostic data.

### BACKGROUND

Many unfavorable complications may evolve from or be caused by a health condition or a therapy. The probability of each complication being caused by a health condition such as a particular disease may vary drastically due to different patients' medical histories. Thus, it is required to take the personal medical history into account when performing medical treatment on a patient. However, a database for storing medical records in a private clinic may not have sufficient medical history of the patients.

### SUMMARY

Therefore, an object of the present disclosure is to provide a method for integrating diagnostic data from different databases.

According to one aspect of the present disclosure, a method for integrating diagnostic data is to be implemented by an electronic device.

The method includes steps of:

reading, from a first database, a plurality of first medical records each including a first identity information and a first medical history that corresponds to the first identify information, and reading, from a second database, a plurality of second medical records each including a second identity information and a second medical history that corresponds to the second identity information; and

determining whether any of the first and second medical records satisfies a user defined condition; and

when the determination made above is affirmative, generating an integrated medical information indicating each of the first and second medical records that satisfies the user defined condition.

### BRIEF DESCRIPTION OF THE DRAWINGS

Other features and advantages of the present disclosure will become apparent in the following detailed description of the embodiments with reference to the accompanying drawings, of which:

FIG. 1 is a schematic block diagram of an electronic device for implementing a method for integrating diagnostic data read from two databases according to an embodiment of the present disclosure; and

FIG. 2 is a flow chart illustrating the method for integrating diagnostic data according to an embodiment of the present disclosure.

### DETAILED DESCRIPTION

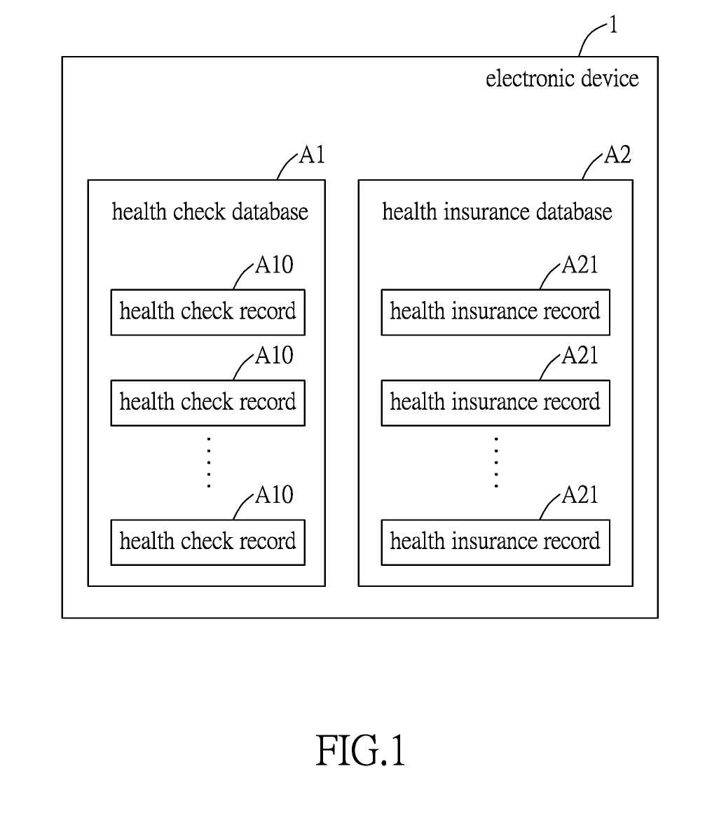

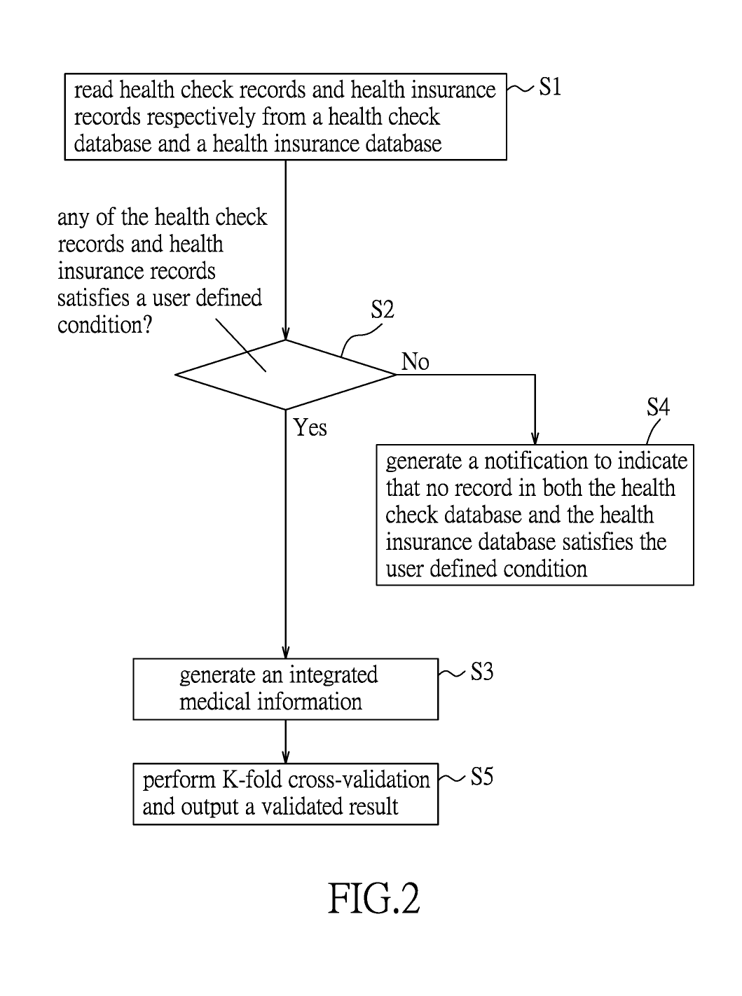

Referring to FIGS. 1 and 2, a method for integrating diagnostic data is to be implemented by an electronic device 1. The electronic device 1 can be a smartphone, a notebook computer or a desktop computer, and the present disclosure is not limited in this respect.

In step S1 of the method, the electronic device 1 reads a plurality of first medical records from a first database and a plurality of second medical records from a second database. In this embodiment, the first database is a health check database A1, whose data originate from a private clinic; and the second database is a health insurance database A2, whose data originate from, for example, a government unit and may be collected from a plurality of hospitals and/or clinics. Note that the present disclosure is not limited in the sources of the data contained in the databases. As shown in FIG. 1, the health check database A1 and the health insurance database A2 are stored in a storage device (not shown) of the electronic device 1 in this embodiment. However, the first and second databases may be stored in different remote servers in other embodiments as long as the first and second databases are accessible by the electronic device 1, and the present disclosure is not limited in this respect.

In this embodiment, the health check database A1 contains the plurality of first medical records, and each of the first medical records is a health check record A10. Each first medical record includes a first identity information and a first medical history corresponding to the first identity information. The first medical history of each of the first medical records includes a plurality of first diagnostic entries each indicating a health-related item and a historical diagnostic result corresponding to the health-related item indicated by the first diagnostic entry. In this embodiment, the first identity information is a health check identity information which includes, e.g., a medical record number, a name, a gender, an age and a residential location of a corresponding patient. The medical record number may be a serial number given by a clinic staff for the corresponding patient who sees a doctor for the first time at the private clinic. An example of a health check record A10 is illustrated in Table 1 below but the disclosure is not limited to this example.

TABLE 1
Health Check Record A10
Identity information

| | |
|---|---|
| Medical Record Number | 10602150008 |
| Name | Wang, John |
| Gender | Male |
| Age | 35 |
| Residential Location | Taipei City |

Medical History

| | | |
|---|---|---|
| Diagnostic | Hypertension | Yes |
| Entries | Diabetes | No |
| | Mellitus | |
| | Hyperlipidemia | No |
| | Acute | Yes |
| | Myocardial | |
| | Infarction | |
| | Percutaneous | No |
| | Coronary | |
| | Intervention | |
| | Stroke | No |
| | Heart Failure | No |

Each of the second medical records includes a second identity information and a second medical history corresponding to the second identity information. In this embodiment, the health insurance database A2 contains the plurality of second medical records and each of the second medical records is a health insurance record A21, the second identity information of each health insurance record is a health insurance identity information and the second medical history of each health insurance record is a health insurance medical history. The health insurance identity information includes, e.g., a transcode information which is a number given by the government for the respective one of the health insurance records A21, a gender and an age of a corresponding patient. The health insurance medical history is similar to the first medical history A10 and may include a plurality of second diagnostic entries each indicating a health-related item and a historical diagnostic result corresponding to the health-related item indicated by the second diagnostic entry. An example of the health insurance record A21 is illustrated in Table 2 below.

TABLE 2
Health insurance Record A21
Identity information

| | |
|---|---|
| Transcode number | 2017021501210 |
| Name | Chen, May |
| Gender | Female |
| Age | 28 |

Health Insurance Medical History

| | | |
|---|---|---|
| Diagnostic | Hypertension | No |
| Entries | Diabetes | Yes |
| | Mellitus | |
| | Hyperlipidemia | No |
| | Acute | No |
| | Myocardial | |
| | Infarction | |
| | Percutaneous | No |
| | Coronary | |
| | Intervention | |
| | Stroke | No |
| | Heart Failure | No |

After step S1 is performed, the electronic device 1 performs step S2, in which the electronic device 1 determines whether any of the first and second medical records (the health check records A10 and the health insurance records A21 in this embodiment) satisfies a user defined condition that includes an identity criterion and a queried health-related criterion. The identity criterion of the user defined condition is directed to the first identity information of the first medical records and the second identity information of the second medical records. The queried health-related criterion of the user defined condition corresponds to one of the health-related items indicated by the first and second diagnostic entries, and is used to find which one(s) of the first and second medical records has a first or second diagnostic entry matching the queried health-related criterion. In this embodiment, the user defined condition is to be applied to filter both the health check records A10 and the health insurance records A21. Specifically, in step S2, the electronic device 1 determines whether any of the first and second medical records has a first or second identity information that satisfies the identity criterion, and a first or second medical history that satisfies the queried health-related criterion. As an example, the identity criterion of the user defined condition includes a limit as to gender and a limit as to age, and the queried health-related criterion of the user defined condition is directed to the first and second diagnostic entries respectively in the health check records A10 and the health insurance records A21. For example, a user defined condition may include an identity criterion including a limit as to gender of “male”, a limit as to age of “from 30 years old to 40 years old” and a queried health-related criterion of “Yes to Hypertension,” which means that a queried target is a male whose age is between 30 and 40 years and who has a health condition of hypertension. When the determination made in step S2 is affirmative, the flow of the method goes to step S3; otherwise, the flow goes to step S4.

In step S4, the electronic device 1 generates a notification for notifying a user of the electronic device 1 that no record in the first and second databases satisfies the user defined condition.

In step S3, the electronic device 1 generates an integrated medical information containing one or more data pieces respectively indicating one or more of the first and second medical records that satisfy the user defined condition (namely, one(s) whose identity information satisfies the identity criterion and one of the first or second diagnostic entries of which matches the queried health-related criterion). Each data piece of the integrated medical information contains, for each of the first medical record(s) indicated thereby, one of the first diagnostic entries, the health related item indicated by which matches the health-related item that the queried health-related criterion corresponds, and at least one of the first diagnostic entries, the health-related item indicated by which is relevant to the health-related item that corresponds to the queried health-related criterion, and for each of the second medical record(s) indicated thereby, one of the second diagnostic entries, the health related item indicated by which matches the health-related item that the queried health-related criterion corresponds, and at least one of the second diagnostic entries, the health-related item indicated by which is relevant to the health-related item that corresponds to the queried health-related criterion. It is noted that relevance between any two health-related items may be predefined in a diagnostic data integrating application stored in the electronic device 1 and such relevance can be modified as desired.

Taking the user defined condition previously described in step S2 as an example, where the user defined condition includes an identity criterion that includes a limit as to gender of “male”, a limit as to age of “from 30 years old to 40 years old” and a queried health-related criterion of “Yes to Hypertension,” the integrated medical information generated in step S3 includes one or more data pieces that altogether indicate every single one of the health check records A10 and health insurance records A21 that completely satisfies the user defined condition of a limit as to gender of “male”, a limit as to age of “from 30 years old to 40 years old” and a queried health-related criterion of “Yes to Hypertension.” Since hypertension is a symptom of stroke, acute myocardial infarction, heart failure or aneurysm, the health-related item of “Hypertension” may be pre-defined as being relevant to each of the health-related items of “Stroke”, “Acute Myocardial Infarction”, “Heart Failure” and “Aneurysm.” Therefore, each data piece of the integrated medical information contains the diagnostic entries corresponding to these health-related items for the respective medical record indicated by the data piece. For instance, the integrated medical information may include one data piece that indicates the medical record of a male person X who is 32 years old and who has hypertension, and that contains a diagnostic entry indicating the heath-related item of “Stroke” and a historical diagnostic result of “No,” a diagnostic entry indicating the heath-related item of “Acute Myocardial Infarction” and a historical diagnostic result of “No,” a diagnostic entry indicating the heath-related item of “Heart Failure” and a historical diagnostic result of “No,” and a diagnostic entry indicating the heath-related item of “Aneurysm” and a historical diagnostic result of “No,” where all four of these diagnostic entries come from in the medical record of the male person X.

Following step S3, the electronic device 1 executes step S5. In step S5, the electronic device 1 performs validation based on the integrated medical information using K-fold cross-validation and outputs a validated result. The validated result indicates a mean validation error. Specifically, K-fold cross-validation is performed by partitioning sample data, i.e., the integrated medical information generated in step S3, into K subsamples. Hereafter, (K−1) of K subsamples are used as analysis data for performing analysis, and the remaining one of the K subsamples is retained as validation data for validating precision of an analysis result obtained from the analysis data. Then, validation is performed K times, each time using a respective subsample as the validation data and the rest of the subsamples as the analysis data. That is to say, K-fold cross-validation analyzes K subsamples partitioned from the sample data with each subsample being analyzed K times.

For example, when the integrated medical information generated in step S3 includes one thousand data pieces respectively correspondingly to one thousand first or second medical records, A10 and K is set as ten in step S5, the integrated medical information will be partitioned into ten sets of subsamples, which are labeled as subsample No. 1, subsample No. 2, . . . , and subsample No. 10. Then, the electronic device 1 performs ten times of validation based on the ten sets of subsamples. Particularly, in a first iteration, subsample No. 10 is used as the validation data and subsamples No. 1 to No. 9 are used as the analysis data. In a second iteration, subsample No. 9 is used as the validation data and subsamples No. 1 to No. 8 and No. 10 are used as the analysis data. In a third iteration, subsample No. 8 is used as the validation data and subsamples No. 1 to No. 7 and No. 9 and No. 10 are used as the analysis data and so forth until each subsample is used as the validation data once. A validation error is obtained upon each iteration, which may be an absolute error or a relative error. The mean validation error indicated by the validated result is an average of the ten validation errors obtained in the ten iterations. Note that other validation methods may be used in other embodiments of this disclosure.

To sum up, the method of the present disclosure is capable of integrating the first medical records read from the first database and the second medical records read from the second database to generate the integrated medical information that indicates every first or second medical record satisfying the user defined condition, and that indicates, for each indicated medical record, at least one diagnostic entry the health-related item indicated thereby being relevant to the health-related item to which the queried health-related criterion of the user defined condition is related. Thus, the method of this disclosure can estimate a probability of occurrence of a health condition which corresponds to a health-related item that is relevant to the health-related item of the queried health-related criterion. Additionally, the integrated medical information is validated using K-fold cross-validation such that a doctor may take the validated result into account while performing medical treatment to the patients.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for integrating diagnostic data, the method to be implemented by an electronic device and comprising steps of:
reading, from a first database, a plurality of first medical records each including a first identity information and a first medical history that corresponds to the first identify information, and reading, from a second database, a plurality of second medical records each including a second identity information and a second medical history that corresponds to the second identity information, and
determining whether any of the first and second medical records satisfies a user defined condition; and
when the determination made above is affirmative, generating an integrated medical information indicating each of the first and second medical records that satisfies the user defined condition.

2. The method as claimed in claim 1, wherein the user defined condition includes an identity criterion directed to the first identity information of the first medical records and the second identity information of the second medical records, and the step of determining whether any of the first and second medical records satisfies a user defined condition includes determining whether the first identity information of any of the first medical records or the second identity information of any of the second medical records satisfies the identity criterion.

3. The method as claimed in claim 2, wherein the first medical history of each of the first medical records includes a plurality of first diagnostic entries each indicating a health-related item and a historical diagnostic result that corresponds to the health-related item indicated by the first diagnostic item; and the second medical history of each of the second medical records includes a plurality of second diagnostic entries each indicating a health-related item and a historical diagnostic result that corresponds to the health-related item indicated by the second diagnostic entry.

4. The method as claimed in claim 3, wherein the user defined condition further includes a queried health-related criterion that corresponds to one of the health-related items indicated by the first or second diagnostic entries;
wherein the step of determining whether any of the first and second medical records satisfies a user defined condition includes determining whether there is any of the first medical records whose first identity information satisfies the identity criterion and one of the first diagnostic entries of whose medical history satisfies the queried health-related criterion, or any of the second medical records whose second identity information satisfies the identity criterion and one of the second diagnostic entries of whose medical history satisfies the queried health-related criterion; and
wherein for each of the first medical records that satisfies the user defined condition, the integrated medical information includes a corresponding data piece indicating the first medical record and containing one of the first diagnostic entries of the first medical record the health-related item indicated by which matches the health-related item that the queried health-related criterion corresponds, and for each of the second medical records that satisfies the user defined condition, the integrated medical information includes a corresponding data piece indicating the second medical record and containing one of the second diagnostic entries of the second medical record the health-related item indicated by which matches the health-related item that the queried health-related criterion corresponds.

5. The method as claimed in claim 3, wherein the user defined condition further includes a queried health-related criterion that corresponds to one of the health-related items indicated by the first or second diagnostic entries;
wherein the step of determining whether any of the first and second medical records satisfies a user defined condition includes determining whether there is any of the first medical records whose first identity information satisfies the identity criterion and one of the first diagnostic entries of whose medical history satisfies the queried health-related criterion, or any of the second medical records whose second identity information satisfies the identity criterion and one of the second diagnostic entries of whose medical history satisfies the queried health-related criterion; and
wherein for each of the first medical records that satisfies the user defined condition, the integrated medical information includes a corresponding data piece indicating the first medical record and containing each of one(s) of the first diagnostic entries of the first medical record the health-related item indicated by which is relevant to the health-related item that the queried health-related criterion corresponds, and for each of the second medical records that satisfies the user defined condition, the integrated medical information includes a corresponding data piece indicating the second medical record and containing each of one (s) of the second diagnostic entries of the second medical record the health-related item indicated by which is relevant to the health-related item that the queried health-related criterion corresponds.

6. The method as claimed in claim 2, wherein each of the first identity information and the second identity information includes a gender and an age, and the identity criterion of the user defined condition includes a limit as to gender and a limit as to age.

7. The method as claimed in claim 1, further comprising a step of:
performing validation based on the integrated medical information using K-fold cross-validation.
